(19) United States
(12) Patent Application Publication
(10) Pub. No.: US 2026/0208230 A1
(43) Pub. Date: Jul. 23, 2026

(54) SUGAR CONTENT SORTING DEVICE FOR SANHUA PLUMS BASED ON NEAR-INFRARED SPECTRUM AND CONTROL METHOD THEREOF

(71) Applicant: Institute of Facility Agriculture, Guangdong Academy of Agricultural Science, Guangzhou (CN)

(72) Inventors: Sai XU, Guangzhou (CN); Huazhong LU, Guangzhou (CN); Xin LIANG, Guangzhou (CN); Weihao ZHENG, Guangzhou (CN); Yuhao HUANG, Guangzhou (CN)

(21) Appl. No.: 19/451,210

(22) Filed: Jan. 16, 2026

(30) Foreign Application Priority Data
Jan. 17, 2025 (CN) 202510077048.0

(51) Int. Cl. B07C 5/342; G01N 21/3563; G01N 21/359; G01N 33/02

(52) U.S. Cl. CPC B07C 5/342; G01N 21/3563; G01N 21/359; G01N 33/025; B07C 2501/009

(57) ABSTRACT

Provided are a sugar content sorting device for Sanhua plums based on a near-infrared spectrum and a control method thereof, relating to the technical field of fruit sugar content detection. The device includes a spectral acquisition device and a fruit sorting device. The spectral acquisition device is fixed to a top of the fruit sorting device. Spectral information of Sanhua plum fruits is acquired by the spectral acquisition device. Based on the acquired spectral information, a control strategy combining a near-infrared spectrum technology and a single-chip microcomputer is used. Rotations of a first baffle and a second baffle are controlled by controlling a first motor and a second motor, thereby completing sugar content sorting control of Sanhua plum fruits.

### CROSS-REFERENCE TO RELATED APPLICATION

This patent application claims the benefit and priority of Chinese Patent Application No. 202510077048.0 filed with the China National Intellectual Property Administration on Jan. 17, 2025, the disclosure of which is incorporated by reference herein in its entirety as part of the application.

### TECHNICAL FIELD

The present disclosure relates to the technical field of fruit sugar content detection, and in particular, to a sugar content sorting device for Sanhua plums based on a near-infrared spectrum and a control method thereof.

### BACKGROUND

A plum belongs to a plant of genus Prunus in the family Rosaceae, and is a deciduous tree famous for its juicy and delicious fruits. A plum fruit is usually round or oval with various colors such as yellow and crimson. The plum pulp is yellow or light green. A Sanhua plum is a plum belonging to a subfamily Prunoideae in the family Rosaceae, and is a deciduous woody plant with simple leaves. The leaf bases usually bear glands. Sanhua plums are favored by consumers for large-sized fruits, excellent flavor, and superior quality.

In a production process of Sanhua plums, sugar content sorting is performed to ensure that consumers can obtain products with the same taste. A sugar content is an important index to measure sweetness of fruits. For Sanhua plums which are famous for sweetness, the sugar content directly affects eating experience of consumers. Through a corresponding sugar content sorting device, Sanhua plums with similar sugar contents can be classified into one category, so that Sanhua plums can be priced according to different sugar contents in sales to meet the needs of different consumers.

Most of the conventional sugar content sorting devices for fruits such as plums have a complex structure, which is difficult to install and maintain. Moreover, a large amount of labor and time are usually required, which is difficult to meet the needs of large-scale production. In addition, the conventional sugar content sorting devices are easily affected by human factors, resulting in an unstable sorting effect, low sugar content sorting accuracy for Sanhua plums, and high device costs and sorting costs, failing to yield favorable economic benefits. Therefore, the present disclosure provides a sugar content sorting device for Sanhua plums based on a near-infrared spectrum and a control method thereof to solve problems existing in the prior art.

### SUMMARY

In view of the foregoing problems, an objective of the present disclosure is to provide a sugar content sorting device for Sanhua plums based on a near-infrared spectrum and a control method thereof, thereby solving problems that the conventional sugar content sorting devices for fruits such as plums have a complex structure, usually require a large amount of labor and time, are difficult to meet the needs of large-scale production, and are easily affected by human factors thus resulting in unstable sorting effects.

In order to achieve the objective of the present disclosure, the present disclosure is achieved by the following technical solution: a sugar content sorting device for Sanhua plums based on a near-infrared spectrum, including a spectral acquisition device and a fruit sorting device, where the spectral acquisition device is fixed to a top of the fruit sorting device, the spectral acquisition device includes a detection box and photoelectric switches symmetrically fixed to inner walls of two sides of the detection box, the detection box is arranged in a square shape and has an opening through which a top is communicated with a bottom, inner walls of other two sides, which are not provided with the photoelectric switches, of the detection box are respectively provided with a light source port and an optical fiber port which are adapted to each other; and the fruit sorting device includes a first sorting box that is fixed to the bottom of the detection box and is in communication with the detection box, and a first motor fixed to an outer wall of the first sorting box, where an output shaft of the first motor penetrates into the first sorting box through a bearing and is fixed with a first baffle, a second sorting box in communication with the first sorting box is fixed to a bottom of the first sorting box, a second motor is fixed to an outer wall of the second sorting box, and an output shaft of the second motor penetrates into the second sorting box through a bearing and is fixed with a second baffle.

In some embodiments, two light blocking plates are symmetrically fixed inside the detection box, the light source port and the optical fiber port are located between the two light blocking plates, and the two light blocking plates are provided with through grooves adapted to the photoelectric switches.

In some embodiments, an incident light source is arranged on a side, adjacent to an outside of the detection box, of the light source port, and the incident light source at a position of the light source port uses an incandescent lamp of 100 W.

In some embodiments, a spectrometer is arranged on a side, adjacent to an outside of the detection box, of the optical fiber port, the spectrometer at a position of the optical fiber port uses a hard trigger mode, and when an external rising edge signal is transmitted to the spectrometer at the position of the optical fiber port, the spectrometer at the position of the optical fiber port starts to acquire a spectrum of a current object.

In some embodiments, two photoelectric switches are through-beam photoelectric switches, when Sanhua plum fruits pass between the two photoelectric switches, the photoelectric switches detect an obstacle and generate a rising edge to be transmitted to the spectrometer at a position of the optical fiber port, and the spectrometer at the position of the optical fiber port acquires current spectral information after receiving a signal.

In some embodiments, a velocity at which the first motor controls the first baffle to rotate to a top is configured to be greater than a falling velocity of Sanhua plum fruits, and a velocity at which the second motor controls the second baffle to rotate to a top is configured to be greater than the falling velocity of the Sanhua plum fruits.

A control method of the sugar content sorting device for Sanhua plums based on the near-infrared spectrum is provided. The control method includes the following steps: allowing Sanhua plum fruits to be sorted to fall into the spectral acquisition device one by one in a free-fall manner through an external feeding device, triggering photoelectric switches on the two sides when the Sanhua plum fruits fall to a central position in the detection box, generating, by the photoelectric switch at this moment, a rising edge signal to be transmitted to a spectrometer at a position of the optical fiber port, allowing a light ray incident into the detection box through the light source port to pass through the Sanhua plum fruits when an external rising edge signal is transmitted to the spectrometer and transmitting a signal to the optical fiber port, starting to acquire current spectral information of the Sanhua plum fruits through the spectrometer after receiving the signal through the optical fiber port, judging and analyzing a sugar content of the Sanhua plum fruits by the spectrometer, transmitting sugar content information to an upper computer by the spectrometer, transmitting the sugar content information to a single-chip microcomputer by the upper computer through serial port communication, making a judgment by the single-chip microcomputer and controlling the first motor and the second motor to respectively drive the first baffle and the second baffle to rotate, allowing the first baffle and the second baffle to be locked in place after rotating to tops and to respectively form sorting channels, and enabling the Sanhua plum fruits to fall through the sorting channels to achieve sorting of the sugar content of the Sanhua plum fruits.

In some embodiments, a number of motors in the fruit sorting device is determined according to a number of sugar content sorting gradients of the Sanhua plum fruits, and the number of sugar content sorting gradients of the Sanhua plum fruits is equal to the number of motors plus one.

The embodiments of the present disclosure achieve the following beneficial effects. According to the present disclosure, spectral information of Sanhua plum fruits is acquired by the spectral acquisition device. Based on the acquired spectral information, a control strategy combining a near-infrared spectrum technology and a single-chip microcomputer is used.

Rotations of a first baffle and a second baffle are controlled by controlling a first motor and a second motor, thereby completing sugar content sorting control of Sanhua plum fruits, achieving automatic sorting, achieving rapid, nondestructive and efficient analysis and identification, reducing human intervention, and ensuring stability of a sorting effect. Compared with a conventional sorting device, the sugar content sorting device has a lower cost, and can improve production efficiency. Moreover, a structure of this technology is simple, requires no complex mechanical structure and control system, and is easily installed and maintained.

### BRIEF DESCRIPTION OF THE DRAWINGS

FIG. 1 is a schematic diagram of a three-dimensional structure of a sugar content sorting device for Sanhua plums according to Embodiment 1 of the present disclosure.

FIG. 2 is a schematic diagram of a three-dimensional structure of a spectral acquisition device according to Embodiment 1 of the present disclosure.

FIG. 3 is a schematic diagram of a three-dimensional structure of a fruit sorting device according to Embodiment 1 of the present disclosure.

FIG. 4 is a schematic diagram of an internal structure of a fruit sorting device according to Embodiment 1 of the present disclosure.

FIG. 5A to FIG. 5C are original spectrograms of a first batch of experiments according to Embodiment 1 of the present disclosure.

FIG. 6A to FIG. 6C are spectrograms of Savitzky-Golay (SG) preprocessing of a first batch of experiments according to Embodiment 1 of the present disclosure.

FIG. 7A to FIG. 7C are spectrograms of Standard Normal Variate (SNV) preprocessing of a first batch of experiments according to Embodiment 1 of the present disclosure.

FIG. 8A to FIG. 8C are scatter diagrams of a predicted value and an actual value of a sugar content of a first batch of Sanhua plums under a Competitive Adaptive Reweighted Sampling-Partial Least Squares Regression (CARS-PLSR) model according to Embodiment 1 of the present disclosure.

FIG. 9A to FIG. 9F are original spectrograms of a second batch of experiments according to Embodiment 1 of the present disclosure.

FIG. 10A to FIG. 10F are spectrograms of SG preprocessing of a second batch of experiments according to Embodiment 1 of the present disclosure.

FIG. 11A to FIG. 11F are spectrograms of SNV preprocessing of a second batch of experiments according to Embodiment 1 of the present disclosure.

FIG. 12A to FIG. 12F are scatter diagrams of a predicted value and an actual value of a sugar content of a second batch of Sanhua plums under a CARS-PLSR model according to Embodiment 1 of the present disclosure.

FIG. 13 is a flowchart of a control method according to Embodiment 2 of the present disclosure.

In the figures: 1 spectral acquisition device; 101 detection block; 102 photoelectric switch; 103 light source port; 104 optical fiber port; 105 light blocking plate; 106 through groove; 2 fruit sorting device; 201 first sorting box; 202 first motor; 203 first baffle; 204 second sorting box; 205 second motor; 206 second baffle.

### DETAILED DESCRIPTION OF THE EMBODIMENTS

In the following, the technical solution in embodiments of the present disclosure will be clearly and completely described with reference to the attached drawings. Apparently, the described embodiments are only some of the embodiments of the present disclosure, rather than all of the embodiments. Based on the embodiments in the present disclosure, all other embodiments obtained by those skilled in the art without paying creative labor fall within the scope of protection of the present disclosure.

A Sanhua plum, as a high-quality fruit, has a good taste and a high nutritional value, and further, has an excellent planting benefit and a broad market prospect. With the improvement of planting techniques and the change of market trends, the Sanhua plum industry is expected to continue to maintain a good development momentum.

Sugar content sorting of Sanhua plums is helpful to improve overall quality and market competitiveness of Sanhua plums. Sanhua plums with a high sugar content are more popular because they are usually sweeter and more delicious. By sorting Sanhua plums with a high sugar content, manufacturers can market the Sanhua plums as high-end products, thereby improving an added value and a profit margin of products. Therefore, sugar content sorting is very important in a production and processing process of Sanhua plums.

During a sugar content sorting process of fruits such as plums, it is necessary to rely on a corresponding sugar content sorting device for sorting. However, most of the existing sugar content sorting devices have problems of complex structure and low accuracy and stability of sugar detection.

### Embodiment 1

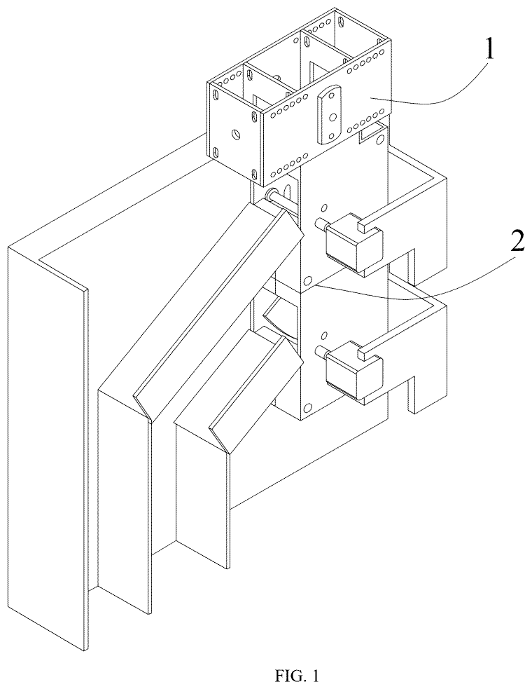

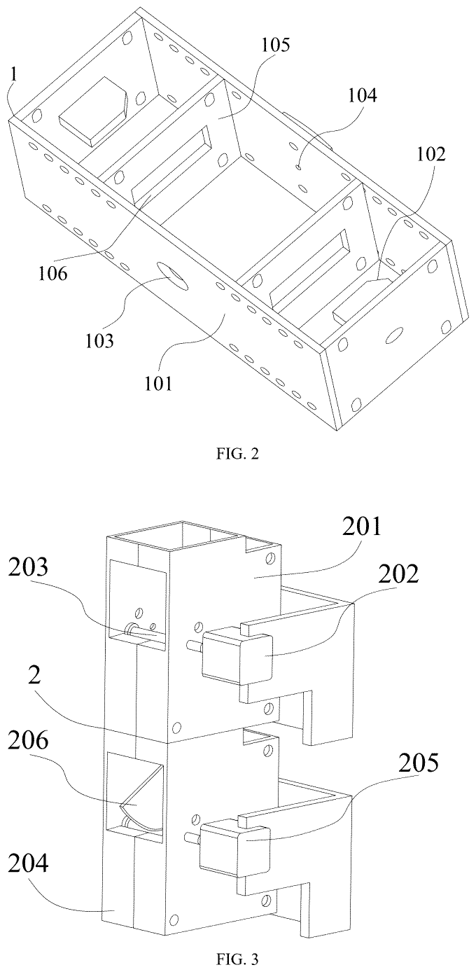

In view of the problems existing in the prior art, refer to FIG. 1, FIG. 2, FIG. 3, and FIG. 4, this embodiment provides a sugar content sorting device for Sanhua plums based on a near-infrared spectrum, which includes a spectral acquisition device 1 which is configured to acquire spectral information of Sanhua plum fruits and a fruit sorting device 2 which is configured to sort a sugar content of the Sanhua plum fruits. The spectral acquisition device 1 is fixed to a top of the fruit sorting device 2 and is in communication with the fruit sorting device 2, so that Sanhua plum fruits can fall from the spectral acquisition device 1 into the fruit sorting device 2.

The spectral acquisition device 1 includes a detection box 101 and photoelectric switches 102. The detection box 101 is arranged in a square shape, in which a top is in communication with a bottom. Two photoelectric switches 102 are arranged. The two photoelectric switches 102 are symmetrically fixed to inner walls on a left side and a right side of the detection box 101 by screws. A front side and a rear side of the detection box 101 are respectively provided with a light source port 103 and an optical fiber port 104. The light source port 103 and the optical fiber port 104 are located in the same central axis and adapted to each other. An external light source is incident to the light source port 103, and the optical fiber port 104 provides a spectral acquisition port for an external spectrometer.

The fruit sorting device 2 includes a first sorting box 201 and a first motor 202. The first sorting box 201 is fixed to a bottom of the detection box 101 through bolts and in communication with the detection box 101. The first motor 202 is fixed to an outer wall of the first sorting box 201 through bolts. An output shaft of the first motor 202 penetrates into the first sorting box 201 through a bearing and is fixed with a first baffle 203. The first baffle 203 is controlled through the first motor 202 to rotate, thereby forming a sorting channel in the first sorting box 201. A second sorting box 204 is fixed to a bottom of the first sorting box 201 through bolts, and the second sorting box 204 is in communication with the first sorting box 201. A second motor 205 is fixed to an outer wall of the second sorting box 204 through bolts. An output shaft of the second motor 205 penetrates into the second sorting box 204 through a bearing and is fixed with a second baffle 206. The second baffle 206 is controlled through the second motor 205 to rotate, thereby forming a sorting channel in the second sorting box 204. Front surfaces of the first sorting box 201 and the second sorting box 204 are both provided with discharge ports, and guide plates are fixed at the discharge ports. The guide plates play a guiding role for the sorted Sanhua plum fruits.

In this embodiment, the Sanhua plum fruits may fall vertically one by one into the detection box 101 through a feeding device, that is, fall in a free-fall manner. When the Sanhua plum fruits pass through a central position in the detection box 101, photoelectric switches 102 on two sides are triggered. In the case of no obstacle (Sanhua plum fruit), light emitted by an emitter may be directly detected by a receiver, and the photoelectric switches 102 may be in the “ON” state. When there is an obstacle (Sanhua plum fruit) passing through a beam path, a beam is blocked or reflected, so that the receiver cannot detect enough light, the photoelectric switches 102 are switched to the “OFF” state, and a rising edge signal is generated.

Light blocking plates 105 that are symmetrically distributed from left to right are fixed inside the detection box 101 through bolts. The light source port 103 and the optical fiber port 104 are located between two light blocking plates 105. The light blocking plate 105 is provided with a through groove 106, and the through groove 106 is adapted to the photoelectric switch 102. The light blocking plate 105 may block influence of a light source on the photoelectric switch 102, so that the photoelectric switch 102 can be triggered as quickly as possible.

An incident light source is arranged on a side, adjacent to an outside of the detection box 101, of the light source port 103. After an experimental test, light sources of 50 W and 75 W cannot pass through the Sanhua plum fruits, so that an effective spectrum cannot be obtained. Therefore, in this embodiment, the incident light source at a position of the light source port 103 uses an incandescent lamp of 100 W.

A spectrometer is arranged on a side, adjacent to the outside of the detection box 101, of the optical fiber port 104. The spectrometer at a position of the optical fiber port 104 uses a hard trigger mode. When an external rising edge signal is transmitted to the spectrometer at the position of the optical fiber port 104, the spectrometer at the position of the optical fiber port 104 starts to acquire a spectrum of a current object.

Two photoelectric switches 102 are through-beam photoelectric switches. Compared with a diffuse-reflective photoelectric switch, the through-beam photoelectric switch has better stability, faster response velocity, and stronger anti-interference ability, and can achieve longer-distance detection. When Sanhua plum fruits pass between the two photoelectric switches 102, the photoelectric switch 102 detects the Sanhua plum fruits and generates an electrical signal, that is, a rising edge signal. The rising edge signal refers to the instant when the signal changes rapidly from a low level (0 volt) to a high level (5 volts). This signal change may be used as a trigger event, so that the spectrometer at position of the optical fiber port 104 starts to perform a spectral acquisition operation and acquire current spectral information of the Sanhua plum fruits, thereby achieving an effect of acquiring a spectrum of the Sanhua plum fruits during a free-fall process of the Sanhua plum fruits.

The spectral information of the plums can be obtained through the judgment and analysis of the spectrometer. The spectral information is first transmitted to an upper computer. The upper computer then obtains the sugar content information of plums through analysis from a CARS-PLSR sugar content model. The upper computer transmits the sugar content information to a single-chip microcomputer through serial port communication. The single-chip microcomputer makes a judgment and controls forward and reverse rotation of a first motor 202 and a second motor 205 to respectively drive a first baffle 203 and the second baffle 206 to rotate. The first baffle 203 and a second baffle 206 are locked in place after rotating to the tops and form sorting channels, so that the Sanhua plum fruits are sorted.

A velocity at which the first motor 202 controls the first baffle 203 to rotate to the top is greater than a falling velocity of Sanhua plum fruits, and a velocity at which the second motor 205 controls the second baffle 206 to rotate to the top is greater than the falling velocity of Sanhua plum fruits, thereby ensuring that a channel has been formed when the Sanhua plum fruits fall, and facilitating sorting of Sanhua plums.

The spectral acquisition device 1 provided by this embodiment is used to perform a spectral acquisition experiment on Sanhua plum fruits. In a first batch of experiments, 120 extra-large fruits are selected with an average mass of 61.2 g and an average diameter of 4.91 cm. In a second batch of experiments, 110 large fruits are selected with an average mass of 49.34 g and an average diameter of 4.33 cm.

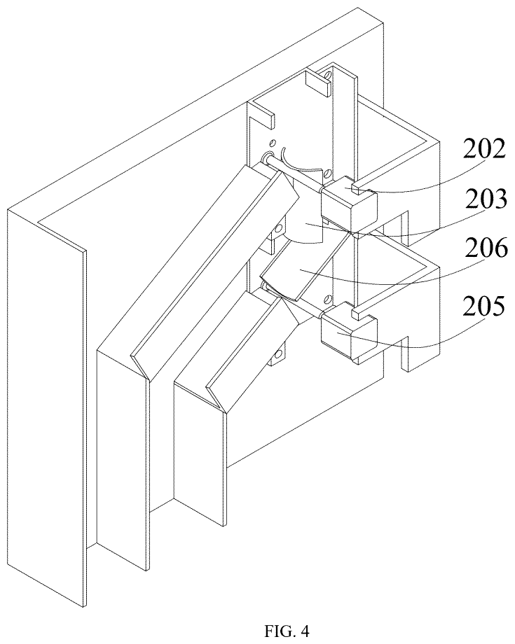

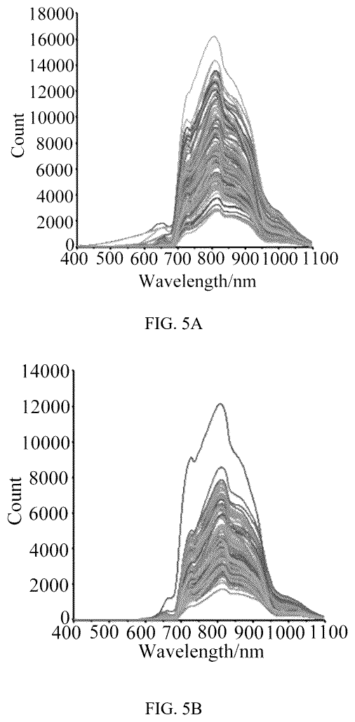

The first batch of experiments mainly analyze integration time, set three gradients for the integration time, perform release at a uniform height, and acquire spectral data. The acquired original spectrogram is shown in FIG. 5A to FIG. 5C (FIG. 5A is 10 ms+5 cm group, FIG. 5B is 15 ms+5 cm group, and FIG. 5C is 20 ms+5 cm group). Table 1 shows changes of the first batch of experimental parameters.

TABLE 1
Changes of the first batch of experimental parameters

| Integration time | Height released during acquisition |
|---|---|
| 10 ms | 5 cm |
| 15 ms | 5 cm |
| 20 ms | 5 cm |

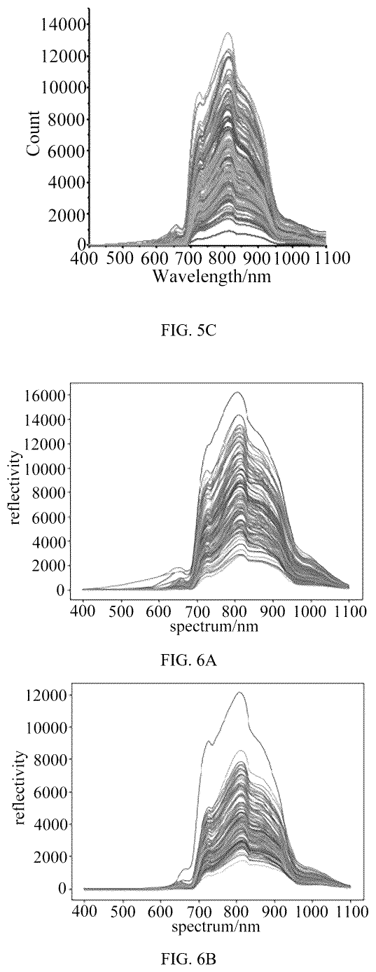

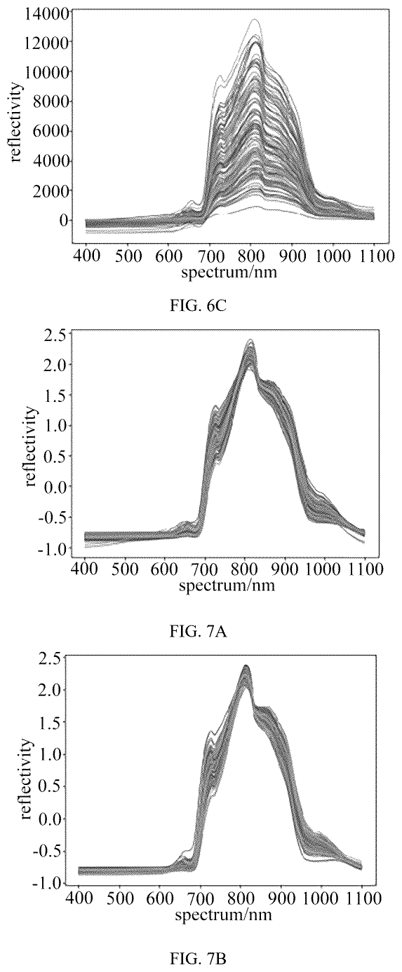

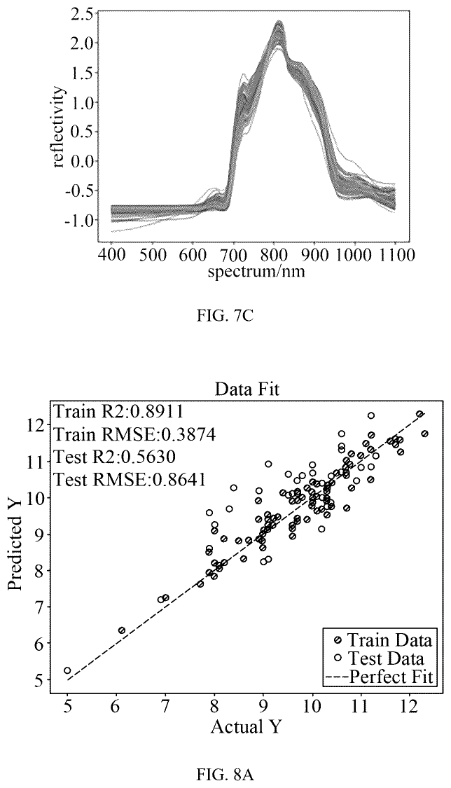

The used preprocessing includes SG smoothing processing algorithm and SNV (Standard Normal Variate) preprocessing. The spectrogram after SG preprocessing is shown in FIG. 6A to FIG. 6C, and the spectrogram after SNV preprocessing is shown in FIG. 7A to FIG. 7C.

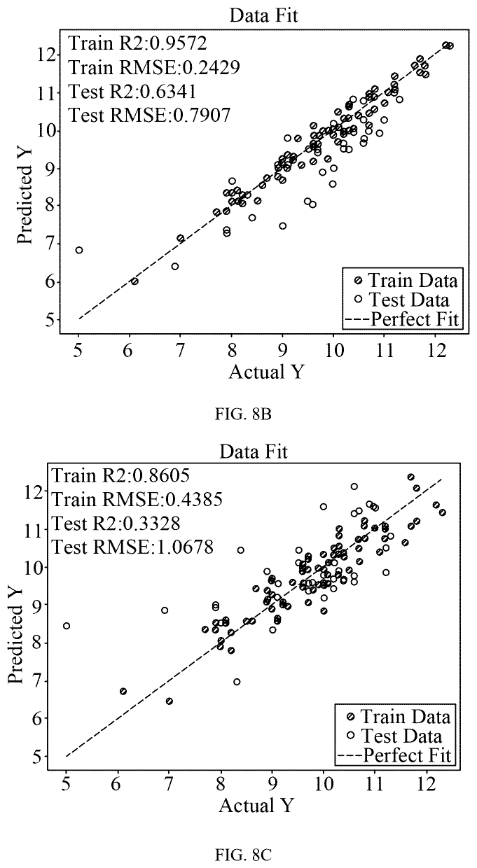

Then, Competitive Adaptive Reweighted Sampling (CARS) feature extraction is performed on the preprocessed spectral data, and a partial least squares regression model is established by using the extracted features. The obtained data are shown in FIG. 8A to FIG. 8C and Table 2 below.

TABLE 2
Data table of a CARS-PLSR model
of the first batch of experiments

| Integration | Distance to a | R-Square | RMSE | R-Square | RMSE |
|---|---|---|---|---|---|
| time | light source | (a) | (a) | (b) | (b) |
| 10 ms | 5 cm | 0.8911 | 0.3874 | 0.563 | 0.8641 |
| 15 ms | 5 cm | 0.9572 | 0.2429 | 0.6341 | 0.7907 |
| 20 ms | 5 cm | 0.8605 | 0.4385 | 0.3328 | 1.0678 |

where a is a test set, and b is a prediction set.

To sum up, it is reasonable to use the integration time of about 15 ms for extra-large Sanhua plum fruits. The longer integration time is used, which may acquire too much invalid information. The shorter integration time is used, which may miss an optimal acquisition interval and cause performance of the model to deteriorate.

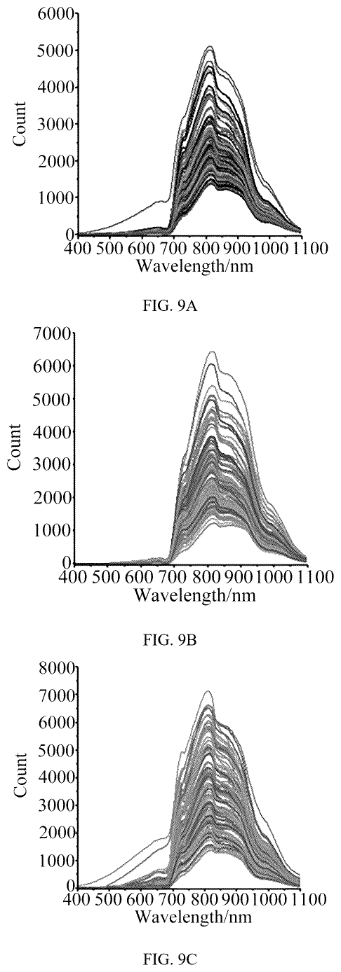

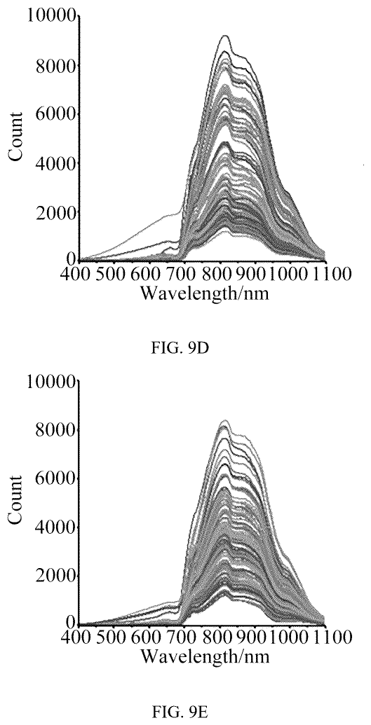

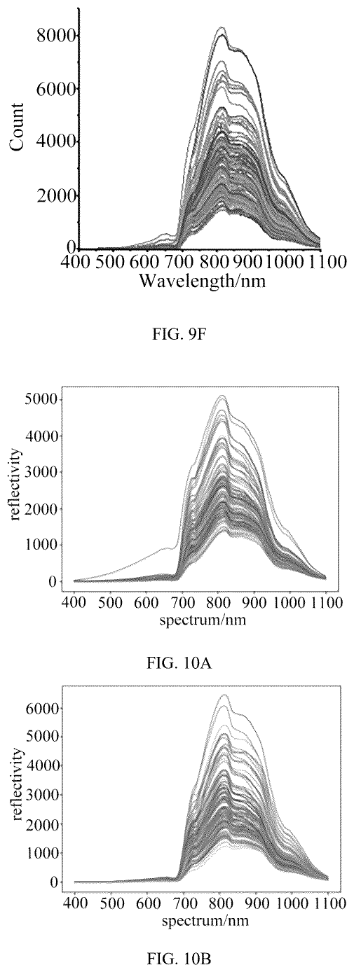

The second batch of experiments mainly analyze the integration time and the release height. The original spectrogram acquired in the second batch of experiments is shown in FIG. 9A to FIG. 9F (FIG. 9A is 8 ms+2.5 cm group, FIG. 9B is 10 ms+2.5 cm group, FIG. 9C is 8 ms+5 cm group, FIG. 9D is 10 ms+5 cm group, FIG. 9E is 8 ms+7.5 cm group, and FIG. 9F is 10 ms+7.5 cm group). Table 3 shows changes of the second batch of experimental parameters.

TABLE 3
Changes of the second batch of experimental parameters

| | |
|---|---|
| Integration time | Height released during acquisition |

| | | | |
|---|---|---|---|
| 8 | ms | 2.5 | cm |
| 10 | ms | 2.5 | cm |
| 8 | ms | 5 | cm |
| 10 | ms | 5 | cm |
| 8 | ms | 7.5 | cm |
| 10 | ms | 7.5 | cm |

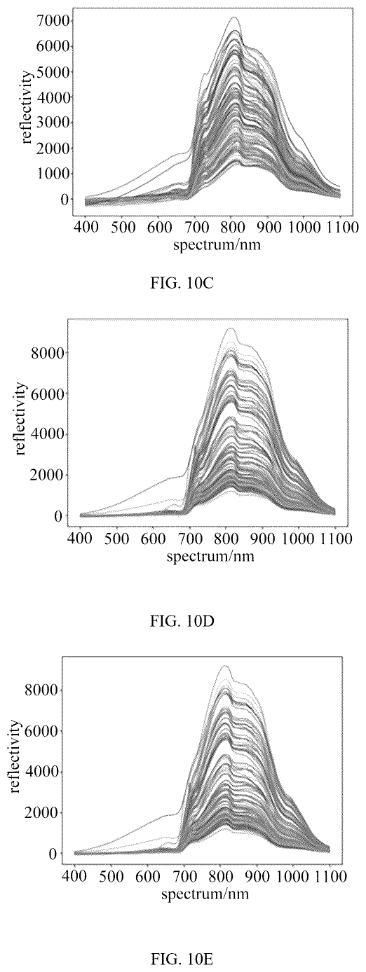

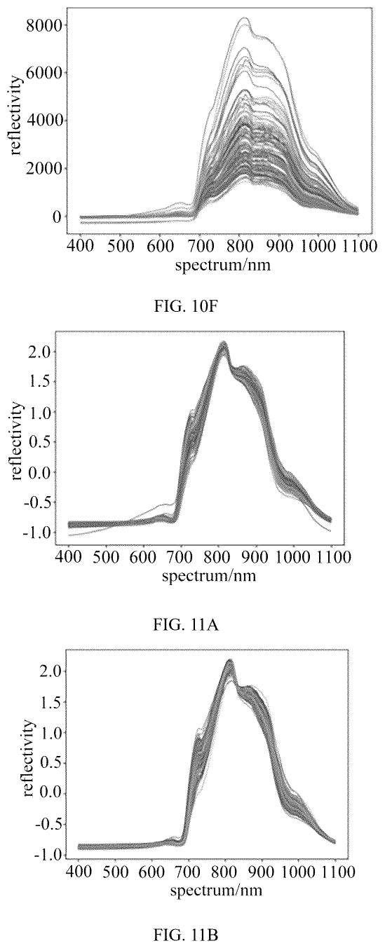

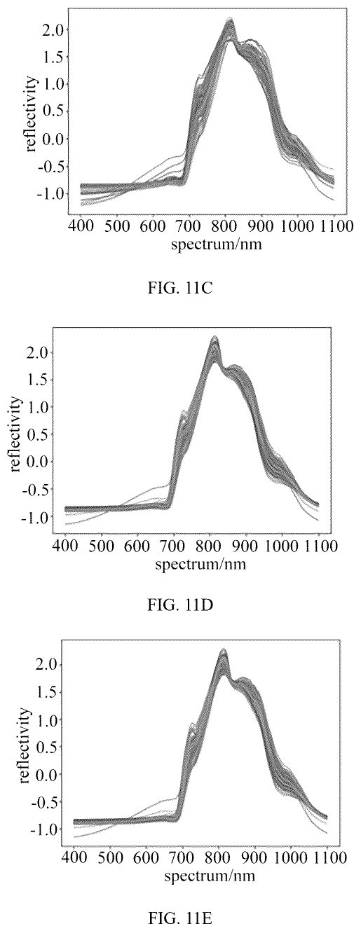

The same preprocessing method as the first batch of experiments is used. The spectrogram after SG preprocessing is shown in FIG. 10A to FIG. 10F, and the spectrogram after SNV preprocessing is shown in FIG. 11A to FIG. 11F.

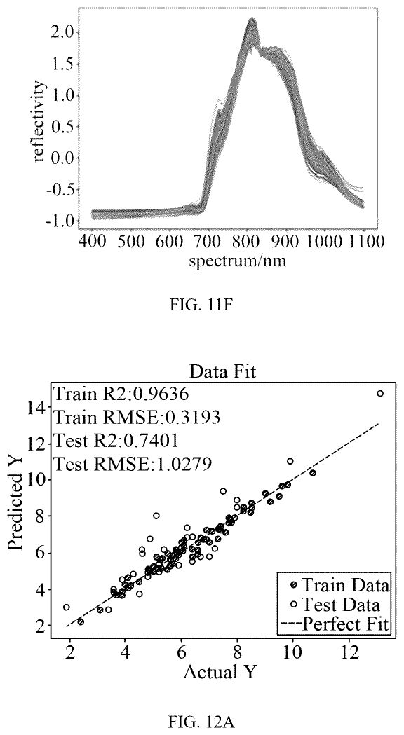

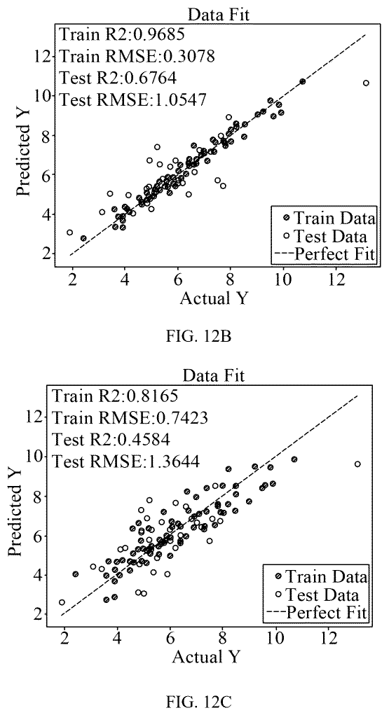

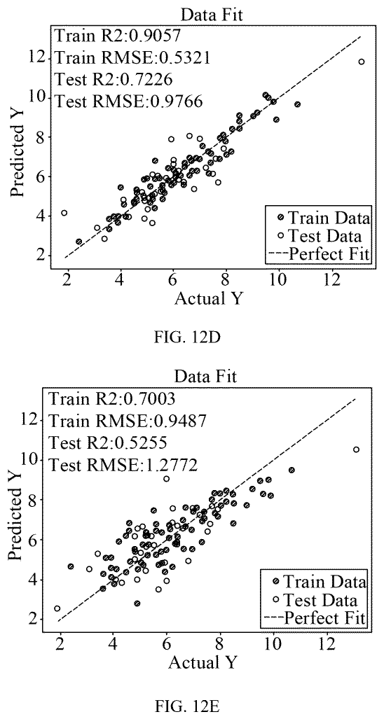

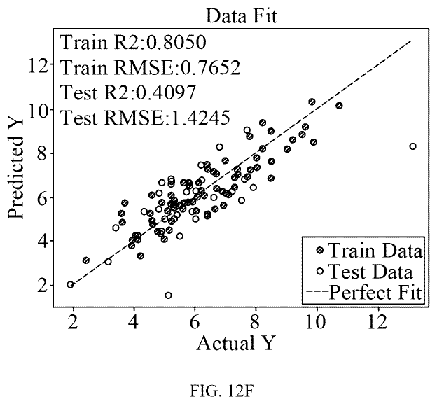

CARS feature extraction is performed on the preprocessed spectral data, and a partial least squares regression model is established by using the extracted features. The obtained data are shown in FIG. 12A to FIG. 12F and Table 4 below.

TABLE 4
Data table of a CARS-PLSR model of the
second batch of Sanhua plum experiments

| Integration | | Distance to a | | R-Square | RMSE | R-Square | RMSE |
|---|---|---|---|---|---|---|---|
| time | | light source | | (a) | (a) | (b) | (b) |
| 8 | ms | 2.5 | cm | 0.9636 | 0.3191 | 0.7401 | 1.0279 |
| 10 | ms | 2.5 | cm | 0.9685 | 0.3078 | 0.6764 | 1.0547 |
| 8 | ms | 5 | cm | 0.8165 | 0.7423 | 0.4584 | 1.3644 |
| 10 | ms | 5 | cm | 0.9057 | 0.5321 | 0.7226 | 0.9766 |
| 8 | ms | 7.5 | cm | 0.7003 | 0.9487 | 0.5255 | 1.2772 |
| 10 | ms | 7.5 | cm | 0.8050 | 0.7652 | 0.4097 | 1.4245 |

where a is a test set, and b is a prediction set.

To sum up, it is reasonable to use the integration time of about 8 ms for large Sanhua plum fruits. The longer integration time is used, which may acquire too much invalid information. The shorter integration time is used, which may miss an optimal acquisition interval and cause performance of the model to deteriorate.

### Embodiment 2

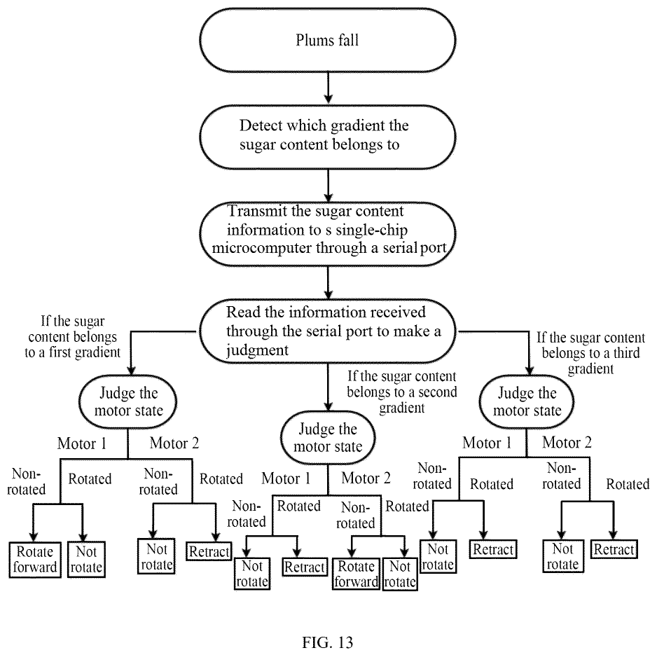

Referring to FIG. 13, according to Embodiment 1, this embodiment provides a control method of the sugar content sorting device for Sanhua plums based on the near-infrared spectrum, including the following steps: Sanhua plum fruits to be sorted fall into the spectral acquisition device 1 one by one in a free-fall manner through an external feeding device, when the Sanhua plum fruits fall to a central position in a detection box 101, photoelectric switches 102 on two sides are triggered, and the photoelectric switch 102 generates a rising edge signal to be transmitted to a spectrometer at a position of an optical fiber port 104, when an external rising edge signal is transmitted to the spectrometer, a light ray incident into the detection box 101 through a light source port 103 passes through the Sanhua plum fruits and the signal is transmitted to the optical fiber port 104, after receiving the signal through the optical fiber port 104, the spectrometer starts to acquire current spectral information of the Sanhua plum fruits, a sugar content of the Sanhua plum fruits is obtained through the judgment and analysis of the spectrometer, sugar content information is then transmitted to an upper computer by the spectrometer, the upper computer then transmits the sugar content information to a single-chip microcomputer through serial port communication, the single-chip microcomputer makes a judgment and controls a first motor 202 and a second motor 205 to respectively drive the first baffle 203 and the second baffle 206 to rotate, the first baffle 203 and the second baffle 206 are locked in place after rotating to tops and respectively form sorting channels, and the Sanhua plum fruits fall through the sorting channels, so that the sugar content of the Sanhua plum fruits is sorted.

In this embodiment, the number of motors in the fruit sorting device 2 is determined according to the number of sugar content sorting gradients of the Sanhua plum fruits, and the number of sugar content sorting gradients of the Sanhua plum fruits is equal to the number of motors plus one. In this embodiment, there are two motors including the first motor 202 and the second motor 205, and the number of sugar content sorting gradients of the Sanhua plum fruits is three. The overall operation logic is as follows.

If the sugar content belongs to a first gradient, rotation flag positions of the first motor 202 and the second motor 205 are detected at this time (1 represents rotated, and 0 represents not rotated). If the first motor 202 is in a rotated state, the first motor does not need to be rotated. If the first motor is in a non-rotated state, the first motor 202 is allowed to rotate forward, and stops after a period of forward rotation. When the first motor rotates to a flag position 1, it indicates that the first motor has been rotated. If the second motor 205 is in a non-rotated state, the second motor does not need to be rotated. If the second motor is in a rotated state, the second motor 205 is allowed to rotate backward, stops after a period of backward rotation, and returns to the starting position.

If the sugar content belongs to a second gradient, rotation flag positions of the first motor 202 and the second motor 205 are still detected at this time (1 represents rotated, and 0 represents not rotated). If the first motor 202 is in a rotated state, the first motor 202 is allowed to rotate backward and returns to the starting position after a period of backward rotation. If the first motor is in a non-rotated state, the first motor does not need to be rotated. If the second motor 205 is in a non-rotated state, the second motor 205 is allowed to rotate forward, and stops after a period of forward rotation. When the second motor rotates to a flag position 1, it indicates that the second motor has been rotated. If the second motor 205 is in a rotated state, the second motor 205 does not need to be rotated.

If the sugar content belongs to a third gradient, rotation flag positions of the first motor 202 and the second motor 205 are detected at this time. As long as the first motor 202 and the second motor 205 are in a rotated state, the first motor and the second motor are allowed to rotate backward. If the first motor 202 and the second motor 205 are in a non-rotated state, the first motor and the second motor do not need to be rotated. In this way, rotation control of the first baffle 203 and the second baffle 206 is completed, thereby enabling plums (Sanhua plum fruits) to be sorted.

The basic principle, main features and advantages of the present disclosure have been shown and described above. It should be understood by those skilled in the art that the present disclosure is not limited by the foregoing embodiments. The principles of the present disclosure are described in the foregoing embodiments and descriptions. Without departing from the spirit and scope of the present disclosure, there may be various changes and improvements in the present disclosure, which fall within the claimed scope of the present disclosure. The claimed scope of the present disclosure is defined by the appended claim and their equivalents.

What is claimed is:

1. A sugar content sorting device for Sanhua plums based on a near-infrared spectrum, comprising a spectral acquisition device (1) and a fruit sorting device (2), wherein the spectral acquisition device (1) is fixed to a top of the fruit sorting device (2), the spectral acquisition device (1) comprises a detection box (101) and photoelectric switches (102) symmetrically fixed to inner walls of two sides of the detection box (101), the detection box (101) is arranged in a square shape and has an opening through which a top is communicated with a bottom, inner walls of other two sides, which are not provided with the photoelectric switches (102), of the detection box (101) are respectively provided with a light source port (103) and an optical fiber port (104) which are adapted to each other; and
the fruit sorting device (2) comprises a first sorting box (201) that is fixed to the bottom of the detection box (101) and is in communication with the detection box, and a first motor (202) fixed to an outer wall of the first sorting box (201), wherein an output shaft of the first motor (202) penetrates into the first sorting box (201) through a bearing and is fixed with a first baffle (203), a second sorting box (204) in communication with the first sorting box is fixed to a bottom of the first sorting box (201), a second motor (205) is fixed to an outer wall of the second sorting box (204), and an output shaft of the second motor (205) penetrates into the second sorting box (204) through a bearing and is fixed with a second baffle (206);
wherein a spectrometer is arranged on a side, adjacent to an outside of the detection box (101), of the optical fiber port (104), the spectrometer at a position of the optical fiber port (104) uses a hard trigger mode, and when an external rising edge signal is transmitted to the spectrometer at the position of the optical fiber port (104), the spectrometer at the position of the optical fiber port (104) starts to acquire a spectrum of a current object, two photoelectric switches (102) are through-beam photoelectric switches, when Sanhua plum fruits pass between the two photoelectric switches (102), the photoelectric switches (102) detect an obstacle and generate a rising edge to be transmitted to the spectrometer at the position of the optical fiber port (104), and the spectrometer at the position of the optical fiber port (104) acquires current spectral information after receiving a signal, a velocity at which the first motor (202) controls the first baffle (203) to rotate to a top is configured to be greater than a falling velocity of the Sanhua plum fruits, and a velocity at which the second motor (205) controls the second baffle (206) to rotate to a top is configured to be greater than the falling velocity of the Sanhua plum fruits.

2. The sugar content sorting device for Sanhua plums based on the near-infrared spectrum according to claim 1, wherein two light blocking plates (105) are symmetrically fixed inside the detection box (101), the light source port (103) and the optical fiber port (104) are located between the two light blocking plates (105), and the two light blocking plates (105) are provided with through grooves (106) adapted to the photoelectric switches (102).

3. The sugar content sorting device for Sanhua plums based on the near-infrared spectrum according to claim 1, wherein an incident light source is arranged on a side, adjacent to the outside of the detection box (101), of the light source port (103), and the incident light source at a position of the light source port (103) uses an incandescent lamp of 100 W.

4. A control method applied to the sugar content sorting device for Sanhua plums based on the near-infrared spectrum according to claim 1, comprising the following steps: allowing the Sanhua plum fruits to be sorted to fall into the spectral acquisition device (1) one by one in a free-fall manner through an external feeding device, triggering photoelectric switches (102) on the two sides when the Sanhua plum fruits fall to a central position in the detection box (101), generating, by the photoelectric switch (102) at this moment, a rising edge signal to be transmitted to the spectrometer at the position of the optical fiber port (104), allowing a light ray incident into the detection box (101) through the light source port (103) to pass through the Sanhua plum fruits when the external rising edge signal is transmitted to the spectrometer and transmitting a signal to the optical fiber port (104), starting to acquire current spectral information of the Sanhua plum fruits through the spectrometer after receiving the signal through the optical fiber port (104), judging and analyzing a sugar content of the Sanhua plum fruits by the spectrometer, transmitting sugar content information to an upper computer by the spectrometer, transmitting the sugar content information to a single-chip microcomputer by the upper computer through serial port communication, making a judgment by the single-chip microcomputer and controlling the first motor (202) and the second motor (205) to respectively drive the first baffle (203) and the second baffle (206) to rotate, allowing the first baffle (203) and the second baffle (206) to be locked in place after rotating to tops and to respectively form sorting channels, and enabling the Sanhua plum fruits to fall through the sorting channels to achieve sorting of the sugar content of the Sanhua plum fruits.

5. The control method of the sugar content sorting device for Sanhua plums based on the near-infrared spectrum according to claim 4, wherein a number of motors in the fruit sorting device (2) is determined according to a number of sugar content sorting gradients of the Sanhua plum fruits, and the number of sugar content sorting gradients of the Sanhua plum fruits is equal to the number of motors plus one.

6. The control method according to claim 4, wherein two light blocking plates (105) are symmetrically fixed inside the detection box (101), the light source port (103) and the optical fiber port (104) are located between the two light blocking plates (105), and the two light blocking plates (105) are provided with through grooves (106) adapted to the photoelectric switches (102).

7. The control method according to claim 6, wherein a number of motors in the fruit sorting device (2) is determined according to a number of sugar content sorting gradients of the Sanhua plum fruits, and the number of sugar content sorting gradients of the Sanhua plum fruits is equal to the number of motors plus one.

8. The control method according to claim 4, wherein an incident light source is arranged on a side, adjacent to the outside of the detection box (101), of the light source port (103), and the incident light source at a position of the light source port (103) uses an incandescent lamp of 100 W.

9. The control method according to claim 8, wherein a number of motors in the fruit sorting device (2) is determined according to a number of sugar content sorting gradients of the Sanhua plum fruits, and the number of sugar content sorting gradients of the Sanhua plum fruits is equal to the number of motors plus one.
